(19) United States
(12) Patent Application Publication
(10) Pub. No.: US 2010/0004738 A1
(43) Pub. Date: Jan. 7, 2010

(54) DRUG DELIVERY COATING FOR USE WITH A MEDICAL DEVICE AND METHODS OF TREATING VASCULAR INJURY

(75) Inventors: Steve A. Herweck, Nashua, NH (US); Paul Martakos, Pelham, NH (US); Geoffrey Moodie, Hudson, NH (US); Theodore Karwoski, Hollis, NH (US); Trevor Carlton, Hudson, NH (US)

Correspondence Address: LAHIVE & COCKFIELD, LLP;FLOOR 30, SUITE 3000, ONE POST OFFICE SQUARE, BOSTON, MA, 02109, US

(73) Assignee: ATRIUM MEDICAL CORPORATION, Hudson, NH (US)

(21) Appl. No.: 11/919,315

(22) PCT Filed: Apr. 28, 2006
(86) PCT No.: PCT/US2006/016502

§ 371 Date: Sep. 17, 2009

Related U.S. Application Data
(60) Provisional application No. 60/676,007, filed on Apr. 29, 2005.
(60) Provisional application No. 60/675,992, filed on Apr. 29, 2005.

(51) Int. Cl. A61F 2/82; A61F 2/00; A61K 31/439; A61M 25/00; A61B 17/03

(52) U.S. Cl. 623/1.42; 424/423; 514/291; 604/523; 606/151

(57) ABSTRACT

The present inventions provide various embodiments of methods for one or more of treating vascular injury, neointima proliferation and/or local inflammation in a mammal by locally administering therapeutic compound comprising a mTOR targeting compound and a calcineurin inhibitor. In various aspects, the therapeutic compound comprises a bio-absorbable carrier component carrier component at least partially formed of a cellular uptake inhibitor and a cellular uptake enhancer, a mTOR targeting compound and a calcineurin inhibitor. In various aspects, the present invention provides for controlled delivery, which is at least partially characterized by total and relative amounts of a cellular uptake inhibitor and cellular uptake enhancer in a bio-absorbable carrier component.

### CROSS-REFERENCE TO RELATED APPLICATIONS

The present application claims the benefit of and priority to copending U.S. Provisional Application No. 60/676,007, filed Apr. 29, 2005, and U.S. Provisional Application No. 60/675,992, filed Apr. 29, 2005, the entire disclosures of both of which are herein incorporated by reference.

### BACKGROUND OF THE INVENTION

Often subsequent to an intravascular procedure neointima proliferation and vascular injury remodeling occurs in the blood vessel of man, more specifically in the heart, as well as in vulnerable peripheral blood vessels like the carotid artery, iliac artery, femoral and popliteal arteries. This results in a narrowing of the vessel lumen, causing restricted flow and pre-disposing to an-ischemic event.

Although, some recently published clinical studies have suggested that selected patients may benefit from the administration of a single sirolimus compound over a period of time systemically (oral) to help control cellular neointimal proliferation caused by stenting, such oral use of single sirolimus compounds have not yet demonstrated adequate or consistent reduction in neointimal proliferation, even with daily use, relative to that achieved with a drug eluting stent.

Medical devices such as coronary stents coated with various forms of drug eluting coatings containing sirolimus drugs have shown promise at controlling vascular wall proliferation following vascular injury and/or vascular reperfusion procedures such as balloon angioplasty and/or mechanical stent deployment.

In many patients a vascular injury location develops into a narrowed or stenotic region, restricting flow and predisposing the vessel to a major thrombotic event, most commonly known as a heart attack (clot occlusion) or blood flow occlusion in the arm or leg, commonly referred to as peripheral occlusion. Once an occluded blood vessel has been “opened up” and/or mechanically cleared of the occluding thrombus which has formed in this narrowed region of the diseased vessel, this narrowed area must be mechanically altered to increase the cross section flow diameter. Hence, it is now widely accepted clinically that mechanically opening the constricted flow area of the vessel, together with the use of a single compound of sirolimus, delivered locally from a stent, provides the best outcome for minimizing re-stenosis.

Today, a preferred drug eluting format includes application, e.g., coating, of a single sirolimus compound on the surface of a radially expandable metal tube. This is generally called a drug eluting stent. Drugs such as Sirolimus (rapamycin), ABT 578, and paclitaxel have at least experimentally been shown to reduce cellular neointimal proliferation following mechanical injury in an otherwise healthy animal. No known mechanical suppression means has been found to prevent or suppress cellular proliferation from occurring, which left untreated can cause re-stenosis within the vessel lumen within weeks of a vascular injury. Local delivery of a single sirolimus or taxol compound has been shown to be the most effective means to minimize uncontrolled cellular proliferation after vascular injury. Therefore, most commercial applications of a drug eluting sirolimus like compound, only include one pharmacologic agent, and one method to treat neointimal proliferation following vascular injury.

### SUMMARY OF THE INVENTION

In spite of the clinical benefits of using a single sirolimus compound locally on a drug eluting stent, experimental studies conducted by and/or on behalf of the inventors have shown that such single compound sirolimus like agents do not fully suppress inflammation and delay healing in and around the localized tissue area of the medical device and its drug eluting location. Lack of endothelial cell coverage during delayed healing induced by rapamycin exhibits a high potential for luminal thrombosis.

As an example, when two separate rapamycin drug eluting stents are placed into an overlapping condition within a rabbit's iliac vessel, the amount of inflammation induced by the overlapping drug coating increases nearly two fold, as determined by the lack of smooth muscle cell proliferation, and massive amounts of fibrin found deposited by the blood at such locations.

Sirolimus like compounds in particular inhibit growth factor driven proliferation of smooth muscle cells following vascular injury. This suggests a potential for therapeutically treating vascular injury vessel disease locally and minimizing restenosis following percutaneous transluminal angioplasty (PTCA). For example, vascular injury events have been shown to cause uncontrolled proliferation of smooth muscle cells in man. Vascular injury also results from endothelial cell disruption and vascular wall injury induced by mechanical means, such as during balloon angioplasty to radially expand the vessel and from stent deployment. Injured blood vessels may self-perpetuate a “chronic” repair process which includes a series of biological events whereby growth factors stimulate proliferation of smooth muscle cells, resulting in internal vessel thickening and excessive vessel narrowing. This may be countered with a sirolimus eluting stent. However, this technique often requires that patients must be kept on powerful anti-platelet Clopidegrel medications and ASA (aspirin) to prevent “in stent” thrombus due to the lack of endothelial cell coverage at these locations as a result of the deployment of a drug eluting stent.

Experimentally in rabbit iliacs, it has been shown that such vascular wall inflammation is not just dependent upon the lack of organized endothelial cell healing, but also is a function of the Rapamycin drug eluting stent inducing or contributing to an inflammatory response as evident by the presence of numerous giant cells and esinophils around the drug eluting stent. The inventors postulate, based on these animal study results, that one consequence of these types of drugs is retarded endothelial cell coverage, and that current drug eluting stents promote more chronic inflammation than would normally be present with a non drug eluting bare metal stent.

The inventors have learned that by locally delivering a single cytokine inhibiting agent like calcineurin inhibiting compounds such as Tacrolimus, and/or Cyclosporine A (CsA) and Cyclosporin derivatives, such compounds (e.g., when used as a second localized drug eluting ingredient) provide a pathway for reducing vascular tissue inflammation, commonly seen following balloon angioplasty, stent deployment and inflammation incurred by a single sirolimus like compound. Studies in rabbit iliacs following vascular injury conducted by and/or on behalf of the inventors show the benefits of using calcineurin inhibiting compounds like Tacrolimus, Cyclosporin A (CsA) and Cyclosporin derivatives, in small animals like the rat, and in rabbits. These particular cytokine inhibiting compounds can effectively reduce inflammation following vascular injury, and following local delivery of sirolimus like compounds, by reducing giant cell and eosinophil formation. Such vascular injury and sirolimus medicated inflammation can be characterized as having excessive giant cell formation and esinophil propagation. Most cytokine and calcineurin inhibiting compounds have been found experimentally in animals not to reduce or exhibit a meaningful anti-proliferative effect (preventing smooth muscle cell proliferation following vascular injury), but rather reduce giant cell and eosinophil propagation found to be a cause of protracted inflammation.

It is therefore a subject of the present inventions, in various aspects, to combine the localized therapeutic administration and use of a mTOR targeting compound, together with a calcineurin inhibiting compound, as a combination treatment therapy, and as part of a drug eluting medical device to improve endothelial cell healing.

In various embodiments, these two compounds create a synergistic biological effect, specific to each compound's distinctive pharmacological benefits, one drug to prevent the proliferation of smooth muscle cells following vascular injury, and the second drug to reduce the inflammation induced by, e.g., the vascular injury. By controlling cellular proliferation and secondarily by effectively reducing the amount of inflammation to the localized area of the vessel being treated with mTOR targeting compound, it is expected in various embodiments that any reduction in inflammation will allow a more rapid and natural endothelial cell healing of the vascular injury. In other words, when a single mTOR targeting compound is delivered locally to the site of the vascular injury to inhibit smooth muscle cell proliferation, use of a second, therapeutic compound such as a calcineurin inhibiting compound like Tacrolimus, or Cyclosporin A and its derivatives, can effectively balance the biological events of modulating smooth muscle cells proliferation and effectively reduce the chronic inflammation so as to encourage a more rapid endothelialization along the injured vascular surface of the vessel.

The present inventions are directed toward therapeutic formulations for local delivery comprising a mTOR targeting compound and a calcineurin inhibitor. In various embodiments, the mTOR targeting compound is of Formula I or a derivative, analog, ester, prodrug, pharmaceutically acceptably salts thereof, or conjugate thereof which has or whose metabolic products have the same mechanism of action. In various embodiments, the calcineurin inhibitor is a compound of Tacrolimus, or a derivative, analog, ester, prodrug, pharmaceutically acceptably salts thereof, or conjugate thereof which has or whose metabolic products have the same mechanism of action or a compound of Cyclosporin or a derivative, analog, ester, prodrug, pharmaceutically acceptably salts thereof, or conjugate thereof which has or whose metabolic products have the same mechanism of action.

In various aspects, the present inventions provide methods for treating vascular injury in a mammal, such as, e.g., a human. In various embodiments, the method of treating vascular injury in a mammal comprises locally administering: (a) a therapeutically effective amount of a mTOR targeting compound for reducing vascular smooth muscle cell proliferation substantially at the site of administration; and (b) a therapeutically effective amount of a calcineurin inhibitor for reduction of inflammation substantially at the site of administration.

In various aspects, the present invention comprises a method of treating vascular injury in a mammal comprising locally administering a therapeutic formulation in a therapeutically effective amount for increasing the rate of endothelial cell formation at the site of vascular injury, the therapeutic formulation comprising a mTOR targeting compound and a calcineurin inhibitor.

In various aspects, the present invention comprises a method of treating two or more of neointima proliferation, giant cell proliferation, eosinophil proliferation and local inflammation in a mammal resulting from the injury to the interior of a vascular vessel of the mammal, comprising locally administering: (a) a therapeutically effective amount of an mTOR targeting compound; and (b) a therapeutically effective amount of a calcineurin inhibitor.

In various aspects, the present inventions provide medical devices having a coating of a therapeutic formulation comprising a mTOR targeting compound and a calcineurin inhibitor, delivering a therapeutic formulation comprising a mTOR targeting compound and a calcineurin inhibitor from a site distal to the portion of the device inserted in a patient, or combinations of one or more of said coating and delivering. In various embodiments, such a coated medical device comprises a coating having a bio-absorbable carrier component, the bio-absorbable carrier component being at least partially formed of a cellular uptake inhibitor and a cellular uptake enhancer, the coating including a therapeutic agents, a mTOR targeting compound and a calcineurin inhibitor. In various embodiments, the coated medical device is implantable in a patient to effect controlled delivery of the therapeutic agent to the patient. In various embodiments the controlled delivery is at least partially characterized by total and relative amounts of the cellular uptake inhibitor and cellular uptake enhancer in the bib-absorbable carrier component.

In various aspects, the present invention provides a method of making a coated medical device, the method comprising providing the medical device; and applying a therapeutic coating comprising a mTOR targeting compound and a calcineurin inhibitor.

In various aspects, the present invention provides a method of making a coated medical device, the method comprising providing the medical device; and applying a coating having a bio-absorbable carrier component, the bio-absorbable carrier component being at least partially formed of a cellular uptake inhibitor and a cellular uptake enhancer, and the coating further including a therapeutic agents a mTOR targeting compound and a calcineurin inhibitor; wherein the coated medical device is implantable in a patient to effect controlled delivery of the therapeutic agent to the patient; and wherein the controlled delivery is at least partially characterized by total and relative amounts of the cellular uptake inhibitor and cellular uptake enhancer in the bio-absorbable carrier component.

In accordance with another embodiment of the present invention, a coated medical device includes a coating having a bio-absorbable carrier component, the bio-absorbable carrier component being at least partially formed of a cellular uptake inhibitor and a cellular uptake enhancer. The coating having solubilized or dispersed therein the therapeutic agents, a mTOR targeting compound and a calcineurin inhibitor. The coated medical device can be implantable in a patient to effect controlled delivery of the therapeutic agents to the patient. The controlled delivery, in various embodiments, is at least partially characterized by total and relative amounts of the cellular uptake inhibitor and cellular uptake enhancer in the bio-absorbable carrier component.

In accordance with various embodiments of the present invention, the bio-absorbable carrier component contains lipids. The bio-absorbable carrier component can be a naturally occurring oil, such as fish oil. The bio-absorbable carrier component can be modified from its naturally occurring state to a state of increased viscosity. The bio-absorbable carrier component can contain omega-3 fatty acids. The bio-absorbable carrier component can also contain alpha-tocopherol.

In accordance with various embodiments of the present invention, methods of making a coated medical device include providing the medical device; a coating is applied, the coating including a mTOR targeting compound and a calcineurin inhibitor. The coated medical device can be implantable in a patient to effect substantially controlled delivery of the coating to the patient.

In various embodiments, substantially controlled delivery of one or more of the therapeutic agents (mTOR compound and calcineurin inhibitor) can be achieved by formulation of the agents as solid particles, e.g., micronized or nanosized particles, in a coating.

In accordance with various embodiments of the present invention, a method of making a coated medical device includes providing the medical device; a coating is applied having a bio-absorbable carrier component, the bio-absorbable carrier component being at least partially formed of a cellular uptake inhibitor and a cellular uptake enhancer. The coating including a mTOR targeting compound and a calcineurin inhibitor, which, in various embodiments, can be solubilized or dispersed in the coating. The coated medical device can be implantable in a patient to effect controlled delivery of the therapeutic agent to the patient. In various embodiments, controlled delivery can be at least partially characterized by total and relative amounts of the cellular uptake inhibitor and cellular uptake enhancer in the bio-absorbable carrier component.

### BRIEF DESCRIPTION OF THE DRAWINGS

The foregoing and other aspects, embodiments, objects, features and advantages of the invention can be more fully understood from the following description in conjunction with the accompanying drawings. In the drawings like reference characters generally refer to like features and structural elements throughout the various figures. The drawings are not necessarily to scale, emphasis instead being placed upon illustrating the principles of the invention.

FIGS. 1A, 1B, 1C, 1D, 1E, 1F, and 1G are perspective illustrations of a variety of medical devices according to aspects of the present invention;

FIG. 2 is a cross-sectional view of the medical device in accordance with one aspect of the present invention;

FIG. 3 is a cross-sectional view of the medical device in accordance with another aspect of the present invention;

FIG. 4 is a flow chart illustrating a method of making the coated medical device of the present invention, in accordance with one embodiment of the present invention;

FIG. 5 is a flow chart illustrating a variation of the method of FIG. 4, in accordance with one embodiment of the present invention;

FIG. 6 is a flow chart illustrating another variation of the method of FIG. 4, in accordance with one embodiment of the present invention;

FIG. 7 is a diagrammatic illustration of a coated medical device in accordance with one embodiment of the present invention.

FIGS. 8-9 are various images of stents described in Examples 1-2.

### DETAILED DESCRIPTION

The present inventions are directed toward therapeutic formulations for local delivery comprising a mTOR targeting compound and a calcineurin inhibitor. In various embodiments, the mTOR targeting compound is a compound of Formula I or a derivative, analog, ester, prodrug, pharmaceutically acceptably salts thereof, or conjugate thereof which has or whose metabolic products have the same mechanism of action. In various embodiments, the calcineurin inhibitor is a compound of Tacrolimus, or a derivative, analog, ester, prodrug, pharmaceutically acceptably salts thereof, or conjugate thereof which has or whose metabolic products have the same mechanism of action or a compound of Cyclosporin or a derivative, analog, ester, prodrug, pharmaceutically acceptably salts thereof, or conjugate thereof which has or whose metabolic products have the same mechanism of action.

In various aspects, the present inventions provide medical devices having a coating of a therapeutic formulation comprising a mTOR targeting compound and a calcineurin inhibitor, delivering a therapeutic formulation comprising a mTOR targeting compound and a calcineurin inhibitor from a site distal to the portion of the device inserted in a patient, or combinations of one or more of said coating, eluting and delivering.

In various aspects, the present inventions provide methods for treating vascular injury in a mammal, such as, e.g., a human. In various embodiments, the methods comprising locally administering a therapeutic formulation in a therapeutically effective amount for increasing the rate of endothelial cell formation at the site of vascular injury, the therapeutic formulation comprising a mTOR targeting compound and a calcineurin inhibitor.

In various embodiments, the methods comprising treating neointima proliferation and local inflammation in a mammal resulting from the injury to the interior of a vascular vessel of the mammal, comprising locally administering: (a) a therapeutically effective amount of an mTOR targeting compound; and (b) a therapeutically effective amount of a calcineurin inhibitor.

### Therapeutically Effective Amount and Dosage Levels

A therapeutically effective amount refers to that amount of a compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective amount refers to that ingredient alone. When applied to a combination, a therapeutically effective amount can refer to combined amounts of the active ingredients that result in the therapeutic effect, whether administered in combination, serially or simultaneously. In various embodiments, where formulations comprise two or more therapeutic agents, such formulations can be described as a therapeutically effective amount of compound A for indication A and a therapeutically effective amount of compound B for indication B, such descriptions refer to amounts of A that have a therapeutic effect for indication A, but not necessarily indication B, and amounts of B that have a therapeutic effect for indication B, but not necessarily indication A.

Actual dosage levels of the active ingredients in a therapeutic formulation of the present invention may be varied so as to obtain an amount of the active ingredients which is effective to achieve the desired therapeutic response without being unacceptably toxic. The selected dosage level will depend upon a variety of pharmacokinetic factors including the activity of the particular therapeutic formulations of the present invention employed, or the ester, salt or amide thereof, the route of administration, the time of administration, the duration of administration, the rate of excretion of the particular compounds being employed, the duration of the treatment, other drugs, compounds and/or materials used in combination with the particular compounds employed, and like factors well known in the medical arts.

mTOR Targeting Compounds

The mammalian target of Rapamycin (mTOR), also named FKBP12 rapamycin-associated protein (FRAP/RAFT/RAPT/SEP) is a serine/threonine protein kinase that is a member of the phosphoinositol kinase-related kinase (PIKK) family. mTOR plays a critical role in transducing proliferative signals mediated through the phosphatidylinositol 3 kinase (PI3K)/protein kinase B (Akt) signaling pathway. mTOR is a protein kinase that plays a key role in mediating the downstream signaling events associated with mitogenic growth factors and cytokines in smooth muscle cells and T lymphocytes. These events can include phosphorylation of p27, phosphorylation of p70 s6 kinase and phosphorylation of BP-1. By targeting mTOR, inhibition of signal(s) required for cell cycle progression, cell growth, and proliferation can occur.

The term “mTOR targeting compound” refers to any compound which modulates mTOR directly or indirectly. An example of an “mTOR targeting compound” is a compound that binds to FKBP 12 to form, e.g., a complex, which in turn inhibits phosphoinostide (PI)-3 kinase, that is, mTOR. In various embodiments, mTOR targeting compounds inhibit mTOR. Suitable mTOR targeting compounds include, for example, rapamycin and its derivatives, analogs, prodrugs, esters and pharmaceutically acceptable salts. In one embodiment, rapamycin derivatives include, for example, sirolimus, 40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 16-pent-2-ynyloxy-32(S)-dihydrorapamycin and TAFA-93. In another embodiment, the rapamycin derivatives can include compounds of formula (I):

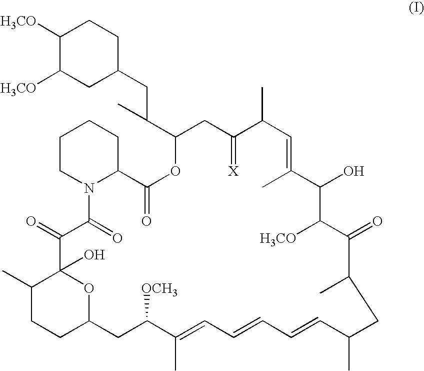

wherein

### X is O or (H,OH).

### Calcineurin Inhibitors

Calcineurin is a serine/threonine phospho-protein phosphatase and is composed of a catalytic (calcineurin A) and regulatory (calcineurin B) subunit (about 60 and about 18 kDa, respectively). In mammals, three distinct genes (A-alpha, A-beta, A-gamma) for the catalytic subunit have been characterized, each of which can undergo alternative splicing to yield additional variants. Although mRNA for all three genes appears to be expressed in most tissues, two isoforms (A-alpha and A-beta) are most predominant in brain.

The calcineuron signaling pathway is involved in immune response as well as apoptosis induction by glutamate excitotoxicity in neuronal cells. Low enzymatic levels of calcineurin have been associated with Alzheimers disease. In the heart or in the brain calcineurin also plays a key role in the stress response after hypoxia or ischemia.

Substances which are able to block the calcineurin signal pathway are suitable therapeutic agents for the present invention. Examples of such therapeutic agents include, but are not limited to, FK506, tacrolimus, cyclosporin and include derivatives, analogs, esters, prodrugs, pharmaceutically acceptably salts thereof, and conjugates thereof which have or whose metabolic products have the same mechanism of action. Further examples of cyclosporin include, but are not limited to, naturally occurring and non-natural cyclosporins prepared by total- or semi-synthetic means or by the application of modified culture techniques. The class comprising cyclosporins includes, for example, the naturally Occurring Cyclosporins A through Z, as well as various non-natural cyclosporin derivatives, artificial or synthetic cyclosporin derivatives. Artificial or synthetic cyclosporins can include dihydrocyclosporins, derivatized cyclosporins, and cyclosporins in which variant amino acids are incorporated at specific positions within the peptide sequence, for example, dihydro-cyclosporin D.

Further examples of substances which are able to block the calcineurin signal pathway can include compounds of formula (II), (III) and (IV):

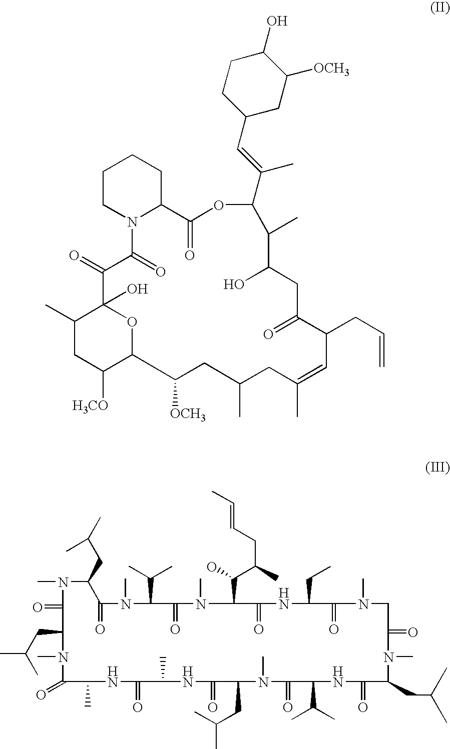

### Vascular Injury

Vascular injury causing intimal thickening can be broadly categorized as being either biologically or mechanically induced. Biologically mediated vascular injury includes, but is not limited to injury attributed to infectious disorders including endotoxins and herpes viruses such as cytomegalovirus; metabolic disorders such as atherosclerosis; and vascular injury resulting from hypothermia, and irradiation. Mechanically mediated vascular injury includes, but is not limited to vascular injury caused by catheterization procedures or vascular scraping procedures such as percutaneous transluminal coronary angioplasty; vascular surgery; transplantation surgery; laser treatment; and other invasive procedures which disrupt the integrity of the vascular intima or endothelium. Generally, neointima formation is a healing response to a vascular injury.

### Inflammatory Response

Wound healing upon vascular injury occurs in several stages. The first stage is the inflammatory phase. The inflammatory phase is characterized by hemostasis and inflammation. Collagen exposed during wound formation activates the clotting cascade (both the intrinsic and extrinsic pathways), initiating the inflammatory phase. After injury to tissue occurs, the cell membranes, damaged from the wound formation, release thromboxane A2 and prostaglandin 2-alpha, potent vasoconstrictors. This initial response helps to limit hemorrhage. After a short period, capillary vasodilatation occurs secondary to local histamine release, and the cells of inflammation are able to migrate to the wound bed. The timeline for cell migration in a normal wound healing process is predictable Platelets, the first response cell, release multiple chemokines, including epidermal growth factor (EGF), fibronectin, fibrinogen, histamine, platelet-derived growth factor (PDGF), serotonin, and von Willebrand factor. These factors help stabilize the wound through clot formation. These mediators act to control bleeding and limit the extent of injury. Platelet degranulation also activates the complement cascade, specifically C5a, which is a potent chemoattractant for neutrophils.

As the inflammatory phase continues, more immune response cells migrate to the wound. The second response cell to migrate to the wound, the neutrophil, is responsible for debris scavenging, complement-mediated opsonization of bacteria, and bacteria destruction via oxidative burst mechanisms (i.e., superoxide and hydrogen peroxide formation). The neutrophils kill bacteria and decontaminate the wound from foreign debris.

The next cells present in the wound are the leukocytes and the macrophages (monocytes). The macrophage, referred to as the orchestrator, is essential for wound healing. Numerous enzymes and cytokines are secreted by the macrophage. These include collagenases, which debride the wound; interleukins and tumor necrosis factor (TNF), which stimulate fibroblasts (produce collagen) and promote angiogenesis; and transforming growth factor (TGF), which stimulates keratinocytes. This step marks the transition into the process of tissue reconstruction, ie, the proliferative phase.

### Cell Proliferation

The second stage of wound healing is the proliferative phase. Epithelialization, angiogenesis, granulation tissue formation, and collagen deposition are the principal steps in this anabolic portion of wound healing. Epithelialization occurs early in wound repair. At the edges of wounds, epidermis immediately begins thickening. Marginal basal cells begin to migrate across the wound along fibrin strands stopping when they contact each other (contact inhibition). Within the first 48 hours the entire wound is epithelialized. Layering of epithelialization is re-established. The depths of the wound at this point contain inflammatory cells and fibrin strands. Aging effects are important in wound healing as many if not most of our problem wounds occur in an older population. For example, cells from older patients are less likely to proliferate and have shorter life spans and cells from older patients are less responsive to cytokines.

Heart disease can be caused by a partial vascular occlusion of the blood vessels that supply the heart, which is preceded by intimal smooth muscle cell hyperplasia. The underlying cause of the intimal smooth muscle cell hyperplasia is vascular smooth muscle injury and disruption of the integrity of the endothelial lining. Intimal thickening following arterial injury can be divided into three sequential steps: 1) initiation of smooth muscle cell proliferation following vascular injury, 2) smooth muscle cell migration to the intima, and 3) further proliferation of smooth muscle cells in the intima with deposition of matrix. Investigations of the pathogenesis of intimal thickening have shown that, following arterial injury, platelets, endothelial cells, macrophages and smooth muscle cells release paracrine and autocrine growth factors (such as platelet derived growth factor, epidermal growth factor, insulin-like growth factor, and transforming growth factor) and cytokines that result in the smooth muscle cell proliferation and migration. T-cells and macrophages also migrate into the neointima. This cascade of events is not limited to arterial injury, but also occurs following injury to veins and arterioles.

### Graizuloinatous Inflammation

Chronic inflammation, or granulomatous inflammation, can cause further complications during the healing of vascular injury. Granulomas are aggregates of particular types of chronic inflamatory cells which form nodules in the millimetre size range. Granulomas may be confluent, forming larger areas. Essential components of a granuloma are collections of modified macrophages, termed epithelioid cells, usually with a surrounding zone of lymphocytes. Epithehoid cells are so named by tradition because of their histological resemblance to epithelial cells, but are not in fact epithelial; they are derived from blood monocytes, like all macrophages. Epithelioid cells are less phagocytic than other macrophages and appear to be modified for secretory functions. The full extent of their functions is still unclear. Macrophages in granulomas are commonly further modified to form multinucleate giant cells. These arise by fusion of epithelioid macrophages without nuclear or cellular division forming huge single cells which may contain dozens of nuclei. In some circumstances the nuclei are arranged round the periphery of the cell, termed a Langhans-type giant cell; in other circumstances the nuclei are randomly scattered throughout the cytoplasm: for example in the foreign body type of giant cell which is formed in response to the presence of other indigestible foreign material in the tissue. Areas of granulomatous inflammation commonly undergo necrosis.

Formation of granulomatous inflammation seems to require the presence of indigestible foreign material (derived from bacteria or other sources) and/or a cell-mediated immune reaction against the injurious agent (type IV hypersensitivity reaction).

### Coated Medical Devices

In various embodiments, the mTOR targeting compound and the calcineurin inhibitor are formulated as a coating for a medical device. In various embodiments, the coating includes a bio-absorbable carrier component. Examples of coated medical devices, include, but are not limited to those implantable in a patient to effect controlled delivery of the therapeutic agents in the coating to the patient.

As utilized herein, the term “bio-absorbable” generally refers to having the property or characteristic of being able to penetrate the tissue of a patient's body. In certain embodiments of the present invention bio-absorption occurs through a lipophilic mechanism. The bio-absorbable substance can be soluble in the phospholipid bi-layer of cells of body tissue, and therefore impact how the bio-absorbable substance penetrates into the cells.

It should be noted that a bio-absorbable substance is different from a biodegradable substance. Biodegradable is generally defined as capable of being decomposed by biological agents, or capable of being broken down by microorganisms or biological processes, in a manner that does not result in cellular uptake of the biodegradable substance. Biodegradation thus relates to the breaking down and distributing of a substance through the patient's body, verses the penetration of the cells of the patient's body tissue. Biodegradable substances can cause inflammatory response due to either the parent substance or those formed during breakdown, and they may or may not be absorbed by tissues.

The phrase “controlled release” generally refers to the release of a biologically active agent in a substantially predictable manner over the time period of several weeks or several months, as desired and predetermined upon formation of the biologically active agent on the medical device from which it is being released. Controlled release includes the provision of an initial burst of release upon implantation, followed by the substantially predictable release over the aforementioned time period.

Therapeutic agents may be delivered to a targeted location in a human utilizing a number of different methods. For example, agents may be delivered nasally, transdermally, intravenously, orally, or via other conventional methods. Delivery may vary by release rate (i.e., quick release or slow release). Delivery may also vary as to how the drug is administered. Specifically, a drug may be administered locally to a targeted area, or administered systemically.

With systemic administration, the therapeutic agent is administered in one of a number of different ways including orally or intravenously to be systemically absorbed by the patient. However, there are drawbacks to systemic delivery of a therapeutic agent, one of which is that high concentrations of the therapeutic agent travels to all portions of the patient's body and can have undesired effects at areas not targeted for treatment by the therapeutic agent. Furthermore, large doses of the therapeutic agent only amplify the undesired effects at non-target areas. As a result, the amount of therapeutic agent that results in application to a specific targeted location in a patient may have to be reduced when administered systemically to reduce complications from toxicity resulting from a higher dosage of the therapeutic agent.

An alternative to the systemic administration of a therapeutic agent is the use of a targeted local therapeutic agent delivery approach. With local delivery of a therapeutic agent, the therapeutic agent is administered using a medical device or apparatus, directly by hand, or sprayed on the tissue, at a selected targeted tissue location of the patient that requires treatment. The therapeutic agent emits, or is otherwise delivered, from the medical device apparatus, and/or carrier, and is applied to the targeted tissue location. The local delivery of a therapeutic agent enables a more concentrated and higher quantity of therapeutic agent to be delivered directly at the targeted tissue location, without having broader systemic side effects. With local delivery, the therapeutic agent that escapes the targeted tissue location dilutes as it travels to the remainder of the patient's body, substantially reducing or eliminating systemic side effects.

Local delivery is often carried out using a medical device as the delivery vehicle. One example of a medical device that is used as a delivery vehicle is a stent. Boston Scientific Corporation sells the Taxus® stent, which contains a polymeric coating for delivering Paclitaxel. Johnson & Johnson, Inc. sells the Cypher® stent which includes a polymeric coating for delivery of Sirolimus.

Targeted local therapeutic agent delivery using a medical device can be further broken into two categories, namely, short term and long term. The short term delivery of a therapeutic agent occurs generally within a matter of seconds or minutes to a few days or weeks. The long term delivery of a therapeutic agent occurs generally within several weeks to a number of months. Typically, to achieve the long term delivery of a therapeutic agent, the therapeutic agent is combined with a delivery agent, or otherwise formed with a physical impediment as a part of the medical device, to slow the release of the therapeutic agent.

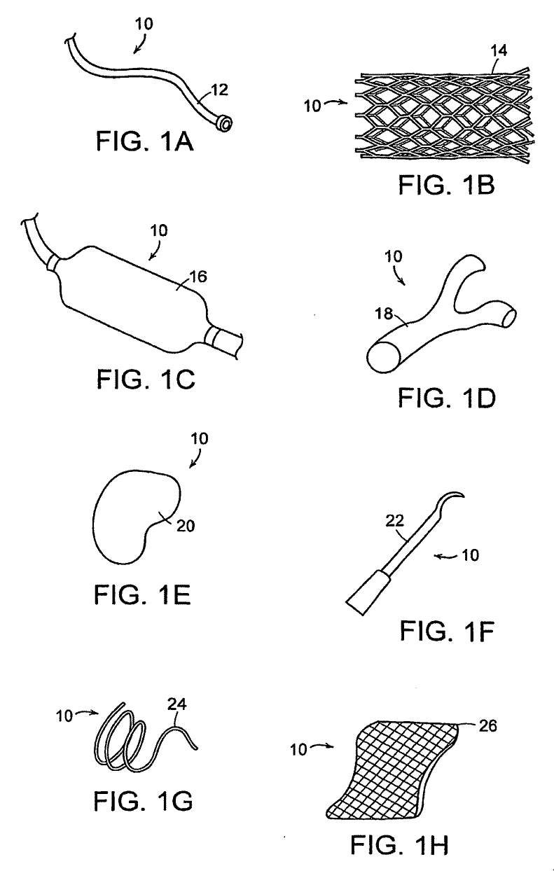

FIGS. 1 through 7, wherein like parts are designated by like reference numerals throughout, illustrate an example embodiment of a coated medical device according to the present invention. Although the present invention will be described with reference to the example embodiments illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

In various embodiments, a coated medical device comprises a therapeutic coating comprising a mTOR targeting compound and a calcineurin inhibitor. The medical device can be any number of devices that have application within a patient. For example, as shown in FIGS. 1A through 1G, the medical device 10 can include a catheter 12 (such as a Foley catheter, suction catheter, urethral catheter, perfusion catheter, PTCA catheter, and the like), a stent 14, a radially expandable device 16 (such as a catheter balloon, or a stent), a graft 18, a prosthesis 20, a surgical tool 22, a suture wire 24, surgical mesh 26 or any other device or tool that makes contact with, or is proximal to, a targeted tissue location within a body cavity or body lumen.

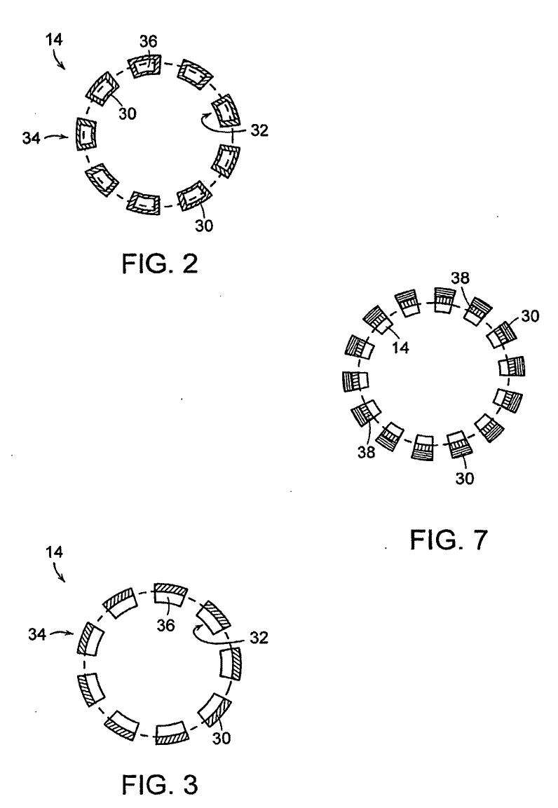

FIG. 2 illustrates various embodiments of the stent 14 having a coating 30 applied thereon in accordance with the present invention. FIG. 3 is likewise an alternative embodiment of the stent 14 having the coating 30 also applied thereon. The coating 30 is applied to the medical device, such as the stent 14, to provide the stent 14 with different surface properties, and also to provide a vehicle for therapeutic applications.

In FIG. 2, the coating 30 is applied on both the interior surface 32 and the exterior surface 34 of the strut 36 forming the stent 14. In other words, the coating 30 in FIG. 2 substantially encapsulates the struts 36 of the stent 14. In FIG. 3, the coating 30 is applied only on the exterior surface 34 of the stent 14, and not on the interior surface 32 of the stent 14. The coating 30 in both configurations is the same coating; the difference is merely the portion of the stent 14 that is covered by the coating 30. One of ordinary skill in the art will appreciate that the coating 30 as described throughout the Description can be applied in both manners shown in FIG. 2 and FIG. 3, in addition to other configurations such as, partially covering select portions of the stent 14 structure. All such configurations are described by the coating 30 reference.

In accordance with various embodiments of the present invention, the medical device 10 includes the coating 30, which is bio-absorbable. The coating 30 has a bio-absorbable carrier component, and can also include the therapeutic agents, a mTOR targeting compound and a calcineurin inhibitor that can also be bio-absorbable. When applied to a medical device such as a stent 14, it is often desirable for the coating to inhibit or prevent restenosis. Restenosis is a condition whereby the blood vessel experiences undesirable cellular remodeling after injury. When a stent is implanted in a blood vessel, and expanded, the stent itself may cause some injury to the blood vessel. The treated vessel typically has a lesion present which can contribute to the inflammation and extent of cellular remodeling. The end result is that the tissue has an inflammatory response to the conditions. Thus, when a stent is implanted, there is often a need for the stent to include a coating that inhibits inflammation, or is non-inflammatory, and prevents restenosis.

In accordance with various embodiments of the present invention, the bio-absorbable carrier component is in the form of a naturally occurring oil. An example of a naturally occurring oil is fish oil or cod liver oil. A characteristic of the naturally occurring oil is that the oil includes lipids, which contributes to the lipophilic action described later herein, that can be helpful in the delivery of therapeutic agents to the cells of the body tissue. In addition, the naturally occurring oil can include omega-3 fatty acids in accordance with several embodiments of the present invention. Omega-3 fatty acids and omega-6 fatty acids are known as essential fatty acids. Omega-3 fatty acids can be further characterized as eicosapentaenoic acid (EPA), docosahexanoic acid (DHA), and alpha-linolenic acid (ALA). Both EPA and DHA are known to have anti-inflammatory effects and wound healing effects within the human body. Alpha-tocopherol can also be incorporated into this coating.

In further detail, the term “bio-absorbable” generally refers to having the property or characteristic of being able to penetrate the tissues of a patient's body. In various embodiments of the present invention, the bio-absorbable coating contains lipids, many of which originate as triglycerides. Triglyceride products such as partially hydrolyzed triglycerides and fatty acid molecules can integrate into cellular membranes and enhance the solubility of drugs into the cell. Whole triglycerides are known not to enhance cellular uptake as well as partially hydrolyzed triglyceride, because it is difficult for whole triglycerides to cross cell membranes due to their relatively larger molecular size. Alpha-tocopherol can also integrate into cellular membranes resulting in decreased membrane fluidity and cellular uptake.

In various embodiments, the bio-absorbable nature of the carrier component and the resulting coating (in the instances where a bio-absorbable therapeutic agent components are utilized) results in the coating 30 being completely absorbed over time by the cells of the body tissue. In various embodiments, there are substantially no substances in the coating, or break down products of the coating, that induce an inflammatory response. The bio-absorbable nature of the coating of the present invention can result in the coating being absorbed, leaving only an underlying delivery or other medical device structure. In various embodiments, there is substantially no foreign body response to the bio-absorbable carrier component.

In various embodiments, the present description makes use of the stent 14 as an example of a medical device that can be coated with the coating 30 of the present invention. However, the present invention is not limited to use with the stent 14. Instead, any number of medical devices, including, but not limited to implantable medical devices, can be coated in accordance with the teachings of the present invention with the described coating 30. Implantation refers to both temporarily implantable medical devices, as well as permanently implantable medical devices.

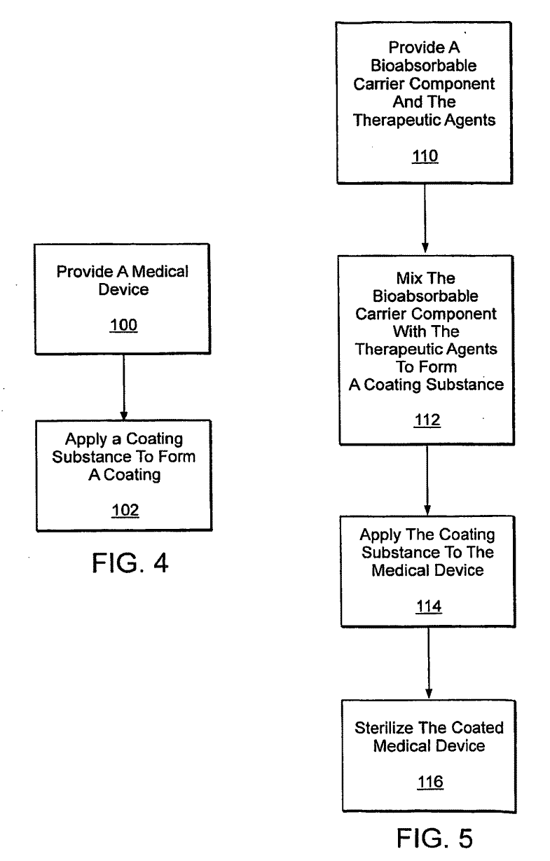

FIG. 4 illustrates various embodiments of methods of making the present invention, in the form of the coated stent 14. The process in various embodiments involves providing a medical device 10, such as the stent 14 (step 100). A coating, comprising a mTOR targeting compound and a calcineurin inhibitor, is then applied to the medical device (step 102). One of ordinary skill in the art will appreciate that this basic method of application of a coating to a medical device such as the stent 14 can have a number of different variations falling within the process described. Depending on the particular application, the medical device 10 with the coating 30 applied thereon can be implanted after the coating 30 is applied, or additional steps such as curing, sterilization, and removal of solvent can be applied to further prepare the medical device 10 and coating 30. Furthermore, if the coating 30 includes a therapeutic agent that requires some form of activation (such as UV light), such actions can be implemented accordingly.

The step of applying a coating substance to form a coating on the medical device such as the stent 14 can include a number of different application methods. For example, the stent 14 can be dipped into a liquid solution of the coating substance. The coating substance can be sprayed onto the stent 14, which results in application of the coating substance on the exterior surface 34 of the stent 14 as shown in FIG. 3. Another application method is painting the coating substance on to the stent 14, which can result in the coating substance forming the coating 30 on the exterior surface 34 as shown in FIG. 3. One of ordinary skill in the art will appreciate that other methods, such as electrostatic adhesion and other application methods, can be utilized to apply the coating substance to the medical device such as the stent 14. Some application methods may be particular to the coating substance and/or to the structure of the medical device receiving the coating. Accordingly, the present invention is not limited to the specific embodiment described herein, but is intended to apply generally to the application of the coating substance to the medical device, taking whatever precautions are necessary to make the resulting coating maintain desired characteristics, e.g., sterility, uniformity, etc.

FIG. 5 is a flowchart illustrating various embodiments of implementation of the methods of FIG. 4. In accordance with the steps illustrated in FIG. 5, a bio-absorbable carrier component is provided along with therapeutic agents, a mTOR targeting compound and a calcineurin inhibitor (step 110). The provision of the bio-absorbable carrier component and the provision of the therapeutic agents can occur individually, or in combination, and can occur in any order or simultaneously. The bio-absorbable carrier component is mixed with the therapeutic agents component (or vice versa) to form a coating substance (step 112). The coating substance is applied to the medical device, such as the stent 14, to form the coating (step 114). The coated medical device is then sterilized using any number of different sterilization processes (step 116). For example, sterilization can be implemented utilizing ethylene oxide, gamma radiation, E beam, or vaporized hydrogen peroxide. One of ordinary skill in the art will appreciate that other sterilization processes can also be applied, and that those listed herein are merely examples of sterilization processes that result in a sterilization of the coated stent, preferably without having a detrimental effect on the coating 30.

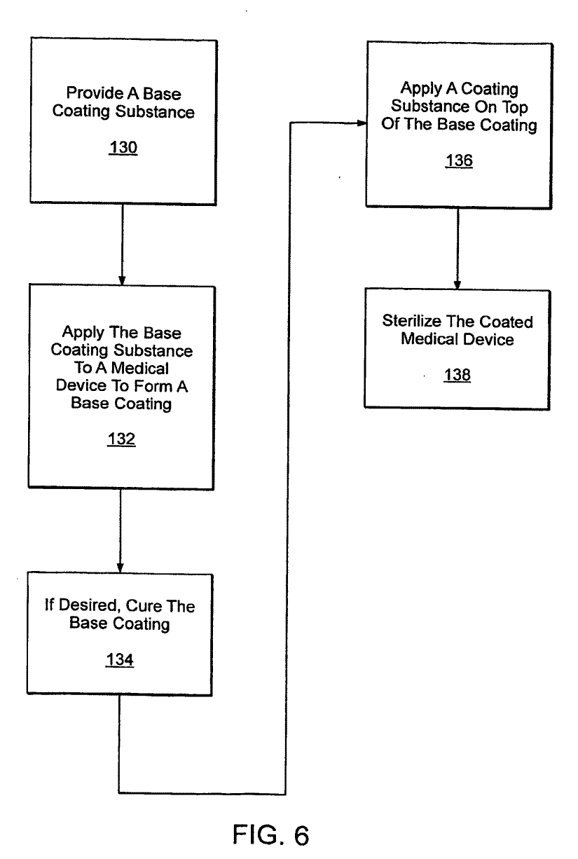

In accordance with various embodiments of the present invention, a surface preparation or pre-treatment 38, as shown in FIG. 7, is provided on a stent 14. More specifically and in reference to the flowchart of FIG. 6, a pre-treatment substance is first provided (step 130). The pretreatment substance is applied to a medical device, such as the stent 14, to prepare the medical device surface for application of the coating (step 132). If desired, the pre-treatment 38 is cured (step 134). Curing methods can include processes such as application of UV light or application of heat to cure the pre-treatment 38. A coating substance is then applied on top of the pre-treatment 38 (step 136). The coated medical device is then sterilized using any number of sterilization processes as previously mentioned (step 138).

FIG. 7 illustrates a stent 14 having two coatings, specifically, the pre-treatment 38 and the coating 30. The pre-treatment 38 can serve as a base or primer for the coating 30. The coating 30 conforms and adheres better to the pre-treatment 38 verses directly to the stent 14, especially if the coating 30 is not heat or UV cured. The pre-treatment can be formed of a number of different materials or substances. In accordance with various embodiments of the present invention, the pre-treatment is formed of a bio-absorbable substance, such as a naturally occurring oil (e.g., fish oil). The bio-absorbable nature of the pre-treatment 38 can result in the pre-treatment 38 ultimately being absorbed by the cells of the body tissue after the coating 30 has been absorbed.

Curing of substances such as fish oil can reduce or eliminate some of the therapeutic benefits of the omega-3 fatty acids, including anti-inflammatory properties and healing properties. However, if the coating 30 contains the bio-absorbable carrier component formed of the oil having the therapeutic benefits, the pre-treatment 38 can be cured to better adhere the pre-treatment 38 to the stent 14, without losing all of the therapeutic benefits resident in the pre-treatment 38, or in the subsequently applied coating 30. In various embodiments, the cured pre-treatment 38 can provide better adhesion for the coating 30 relative to when the coating 30 is applied directly to the stent 14 surface. In various embodiments, the pre-treatment 38, can, despite being cured, remain bio-absorbable.

The pre-treatment 38 can be applied to both the interior surface 32 and the exterior surface 34 of the stent 14, if desired, or to one or the other of the interior surface 32 and the exterior surface 34. Furthermore, the pre-treatment 38 can be applied to only portions of the surfaces 16 and 18, or to the entire surface, if desired.

The application of the coating 30 to the stent 14, or other medical device, can take place in a manufacturing-type facility and subsequently shipped and/or stored for later use. The coating 30 can be applied to the stent 14 just prior to implantation in the patient. The process utilized to prepare the stent 14 will vary according to the particular embodiment desired. In the case of the coating 30 being applied in a manufacturing-type facility, the stent 14 can be provided with the coating 30 and subsequently sterilized in accordance with any of the methods provided herein, and/or any equivalents. The stent 14 can be then packaged in a sterile environment and shipped or stored for later use. When use of the stent 14 is desired, the stent is removed from the packaging and implanted in accordance with its specific design.

In the instance of the coating being applied just prior to implantation, the stent can be prepared in advance. The stent 14, for example, can be sterilized and packaged in a sterile environment for later use. When use of the stent 14 is desired, the stent 14 is removed from the packaging, and the coating substance is applied to result in the coating 30 resident on the stent 14. The coating 30 can result from application of the coating substance by, for example, the dipping, spraying, brushing, swabbing, wiping, or painting methods.

In various embodiments, the present invention provides a coating 30, comprising a mTOR targeting compound and a calcineurin inhibitor, for medical devices, wherein, e.g., the coating can be bio-absorbable.

In various embodiments, the bio-absorbable carrier component itself, in the form of fish oil for example, can provide therapeutic benefits in the form of reduced inflammation, and improved healing, if the fish oil composition is not substantially modified during the process that takes the naturally occurring fish oil and forms it into the coating 30. Some prior attempts to use natural oils as coatings have involved mixing the oil with a solvent, or curing the oil in a manner that destroys the beneficial therapeutic aspects of the oil. The solvent utilized in the coating 30 of the present invention (NMP) does not have such detrimental effects on the therapeutic properties of the fish oil. Thus, the omega-3 fatty acids, and the EPA and DHA substances are substantially preserved in the coating of various embodiments of the present invention. Furthermore, the coating 30 of various embodiments of the present invention is not heat cured or UV light cured to an extent that would destroy all or a substantial amount of the therapeutic benefits of the fish oil.

The coating 30 of various embodiments of the present invention can include the bio-absorbable carrier component in the form of the naturally occurring oil (i.e., fish oil, or any equivalents), which can be absorbed by the cells of a body tissue. For example, there is a phospholipid layer in each cell of the body tissue. The fish oil, and equivalent oils, contain lipids as well. There can be a lipophilic action that results where the lipids are attracted by each other in an effort to escape the aqueous environment surrounding the lipids. Accordingly the lipids attract, the fish oil fatty acids bind to the cells of the tissue, and subsequently alter cell membrane fluidity and cellular uptake. In various embodiments, if there is a therapeutic agent component(s) mixed with the bio-absorbable carrier component, the therapeutic component(s) associated with the fish oil lipids can penetrate the cells.

The lipophilic mechanism enabled by the bio-absorbable lipid based coating 30 of various embodiments of the present invention, can facilitate the uptake of the therapeutic agent by delivery of the therapeutic agent to the cell membrane by the bio-absorbable carrier component. In various embodiments, the therapeutic agent is not freely released into the body fluids, but rather, can be delivered directly to the cells and tissue.

The bio-absorbable nature of various embodiments of the carrier component and the resulting coating (in the instances where a bio-absorbable therapeutic agent component is utilized) can result in the coating 30 being completely absorbed over time by the cells of the body tissue. In various embodiments, there is substantially no break down of the coating into sub parts and substances which induce an inflammatory response that are eventually distributed throughout the body and in some instances disposed of by the body, as is the case with biodegradable coatings. In various embodiments, the bio-absorbable nature of the coating 30 of the present invention can result in the coating being absorbed, leaving only the stent structure, or other medical device structure. In various embodiments, there is substantially no foreign body response to the bio-absorbable carrier component.

Despite action by the cells, in various embodiments of the coating 30 of the present invention can be further configured to release the therapeutic agent component at a rate no faster than a selected controlled release rate over a period of weeks to months. The controlled release rate action can be achieved, e.g., by providing an increased level of alpha-tocopherol (e.g., vitamin E) in the mixture with the fish oil, to create a more viscous, sticky, coating substance that better adheres and lasts for a longer duration on the implanted medical device. The controlled release rate can include an initial burst of release, followed by the sustained multi-week to multi-month period of release. For example, with a greater amount of fatty acids relative to the level of vitamin E, the controlled release rate can be increased. The fatty acids can be found in the oil, and/or fatty acids such as myristic acid can be added to the oil. Thus, the ratio of fatty acids to alpha-tocopherol can be varied in the preparation of the coating 30 to vary the subsequent release rate of the therapeutic agent in a substantially controlled and substantially predictable manner.

The oil can provide a lubricious surface against the vessel walls. As the stent 14 having the coating 30 applied thereon is implanted within a blood vessel, for example, there can be some friction between the stent walls and the vessel walls. This can be injurious to the vessel walls, and increase injury at the diseased vessel location. The use of the naturally occurring oil, such as fish oil, can provide extra lubrication to the surface of the stent 14, which reduces the initial injury. With less injury caused by the stent, there is less of an inflammatory response, and less healing required.

Various aspects and embodiments of the present invention are further described by way of the following Examples. The Examples are offered by way of illustration and not by way of limitation.

### Example 1

Rapamycin/Cypher Study: The Atrium Flyer coated stent loaded with low or high dose sirolimus is implanted in rabbit illiac arteries for 28 days. The Atrium Flyer is compared with bare metal stents, the Atrium Flyer coated with ALPHA-3 without drugs, and Cypher™ drug eluting stent. Histomorphic and histopathologic analyses are then performed.

Atrium Flyer Results

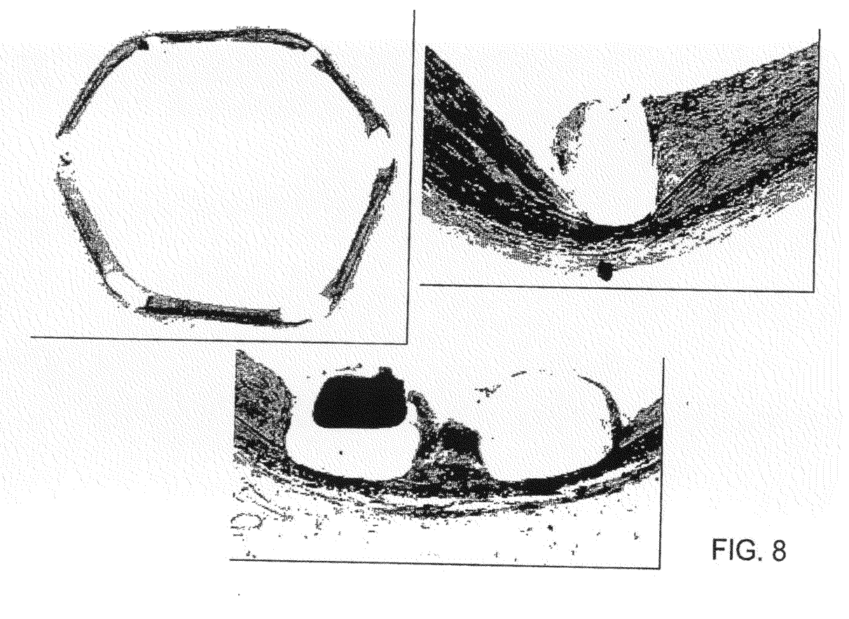

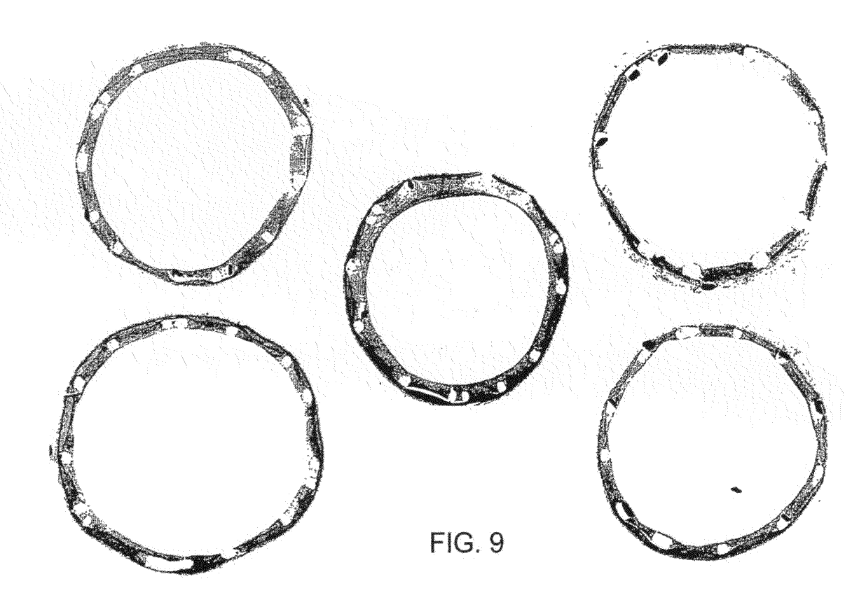

The results are seen in Table 1, Table 2, Chart 1, Chart 2 and FIG. 8. In general, the Atrium Flyer ALPHA-3 coated stent loaded with low or high doses of sirolimus is well tolerated, producing no adverse reaction. Atrium Flyer stents with coating alone produce minimal tissue reactions and are remarkably similar to uncoated bare stainless steel stents. Furthermore, Atrium Flyer stents coated with sirolimus significantly reduce neoinimal growth at 28 days. In addition, although the reduction in intima is accompanied by delayed healing, Atrium drug coated stents are well endothelialized.

Atrium Flyer bare and coated (with and without drug) stents has a minimal arterial injury score; while the Cypher™ stents show a much higher score, more than twice that of the Atrium stents.

The reduction in neointima growth is most significant with Cypher™ stents, however there is evidence delayed healing represented by significant fibrin deposition and minimal to poor endothelialization.

There are a greater number of inflammatory and giant cells seen in Cypher™ stents than exhibited on the Atrium Flyer implants with and or without coating and the drug sirolimus.

Cypher™ stents show at least a 3-fold increase in giant cell reaction and the mosority of stent struts show the presence of eosinophils while Atrium drug eluting stens show only rare eosinophils around stent struts.

TABLE 1
Morphometric comparison of cross sectional vessel area and neointimial
response of polymer-coated, bare and drug eluting stents.

| | EEL | IEL | | | Intimal | | | Medial | |
|---|---|---|---|---|---|---|---|---|---|
| | Area | Area | Lumen Area | Medial Area | Area | Stenosis | Intimal Thick. | Thick. | |
| Treatment Group | mm² | mm² | mm² | mm² | mm² | (%) | mm | mm | Injury Score |
| Bare | 6.02 ± 0.30 | 5.67 ± 0.27 | 4.54 ± 0.24 | 0.35 ± 0.66 | 1.16 ± 0.13 | 19.82 ± 2.45 | 0.16 ± 0.15 | 0.06 ± 0.02 | 0.05 ± 0.04 |
| (n = 8) | | | | | | | | | |
| Polymer | 6.10 ± 0.41 | 5.77 ± 0.40 | 4.67 ± 0.49 | 0.34 ± 0.05 | 1.09 ± 0.12 | 19.13 ± 3.28 | 0.09 ± 0.02 | 0.05 ± 0.01 | 0.07 ± 0.07 |
| (n = 8) | | | | | | | | | |
| Low dose | 5.99 ± 0.44 | 5.63 ± 0.38 | 4.74 ± 0.32 | 0.36 ± 0.09 | 0.97 ± 0.11 | 17.15 ± 1.84 | 0.07 ± 0.01 | 0.05 ± 0.01 | 0.06 ± 0.07 |
| (n = 8) | | | | | | | | | |
| High dose | 6.24 ± 0.25 | 5.90 ± 0.25 | 4.94 ± 0.27 | 0.34 ± 0.05 | 0.99 ± 0.10 | 16.73 ± 1.61 | 0.08 ± 0.03 | 0.06 ± 0.02 | 0.04 ± 0.06 |
| (n = 10) | | | | | | | | | |
| Cypher | 6.75 ± 0.43 | 6.42 ± 0.39 | 5.56 ± 0.39 | 0.33 ± 0.08 | 0.86 ± 0.17 | 13.46 ± 2.67 | 0.05 ± 0.02 | 0.05 ± 0.02 | 0.12 ± 0.08 |
| (n = 8) | | | | | | | | | |

Values represent the means ± SD.
The numbers in parentheses represent total devices.

TABLE 2
Morphometric comparison of polymer and drug effects on vessel compared bare.

| | % struts with | | % | % struts | Struts with | Inflamm. |
|---|---|---|---|---|---|---|
| Treatment Group | Fibrin | Fibrin Score | Endothel. | with RBC | Giant cells | Score |
| Bare (n = 8) | 1.94 ± 4.60 | 0.00 ± 0.00 | 99.88 ± 0.35 | 2.10 ± 3.35 | 1.54 ± 0.86 | 0.20 ± 0.35 |
| Polymer (n = 8) | 0.85 ± 1.18 | 0.00 ± 0.00 | 97.50 ± 5.90 | 1.49 ± 2.53 | 0.66 ± 0.72 | 0.20 ± 0.35 |
| Low dose (n = 8) | 34.12 ± 20.62 | 0.20 ± 0.35 | 97.62 ± 2.67 | 3.46 ± 3.07 | 2.50 ± 1.41 | 0.53 ± 0.41 |
| High dose (n = 10) | 11.99 ± 6.81 | 0.11 ± 0.09 | 97.90 ± 4.58 | 2.86 ± 3.23 | 2.80 ± 0.63 | 0.03 ± 0.10 |
| Cypher (n = 8) | 42.73 ± 10.55 | 0.16 ± 0.35 | 89.00 ± 4.07 | 5.27 ± 8.20 | 6.50 ± 1.69 | 1.75 ± 0.70 |

Values represent the means ± SD.
The numbers in parentheses represent total devices.
Abbreviations:
Endothel. = endothelium,
RBC = red blood cells,
Inflamm. = Inflammation

### Cypher™ Results

The results are seen in Table 1, Table 2, Chart 1, Chart 2 and FIG. 8. Cypher™ stents show delayed healing represented by moderate fibrin deposition, red blood cell infiltration around stent struts, inflammatory cell infiltrate and giant cells. There is severe giant cell reaction around the stent struts and most struts were surrounded by small numbers of eosinophils. The polymer is clearly present on histology as an opaque film around struts. Vessels with Cypher™ implants show incomplete endothelialization with <89% luminal coverage. Intimal growth is the least with Cypher™ stents compared with low- and high-dose eluting Atrium Flyer stents.

### Example 2

Methylprednisolone/Cilostazol/Paclitaxel/Taxus™ The Atrium Flyer stent coated with ALPHA-3 loaded with a high dose sirolimus is implanted in rabbit illiac arteries for 28 days. The Atrium Flyer is compared with the Atrium Flyer coated with ALPHA-3 loaded with low, mid and high doses of paclitaxel, the Atrium Flyer coated with ALPHA-3 and loaded with low, mid and high doses of cilostazol, the Atrium Flyer coated with ALPHA-3 loaded with low, mid and high doses or methylprednilosone, and Taxus™ Express drug eluting stent. Histomorphic and histopathologic analyses are then performed. The results are seen in Table 3, Table 4, and Chart 4.

Results

Atrium high dose paclitaxel and sirolimus stents suppresses in-stent neointimal growth at 28 days similarly to the Taxus™ Express stent. The Atrium high dose paclitaxel and sirolimus stents show (greater) arterial healing at 28 days compared with Taxus™ Express. No reduction in neointima was seen with cilostazol or methlyl prednisolone coated stents.

TABLE 3
Morphometric comparison of cross-sectional vessel areas and neotinimal
response of polymer-coated, bare and drug eluting stents.

| Treatment | EEL Area | IEL Area | Lumen Area | Intimal Area | Stenosis |
|---|---|---|---|---|---|
| Group | mm² | mm² | mm² | mm² | (%) |
| TAXUS ™ | 7.23 ± 0.39 | 6.91 ± 0.32 | 5.77 ± 0.19 | 1.138 ± 0.137 | 16.4 ± 1.2 |
| Express (n = 4) | | | | | |
| Sirolimus High | 6.13 ± 0.15 | 5.80 ± 0.16 | 4.78 ± 0.20 | 1.027 ± 0.036 | 17.7 ± 1.1 |
| dose (n = 4) | | | | | |
| Paclitaxel Low | 5.88 ± 0.41 | 5.58 ± 0.36 | 4.35 ± 0.40 | 1.234 ± 0.197 | 22.2 ± 4.0 |
| dose (n = 8) | | | | | |
| Paclitaxel | 5.56 ± 0.26 | 5.31 ± 0.25 | 4.16 ± 0.27 | 1.152 ± 0.243 | 21.8 ± 5.0 |
| Mid-dose(n = 8) | | | | | |
| Paxlitaxel | 5.63 ± 0.24 | 5.40 ± 0.24 | 4.35 ± 0.22 | 1.048 ± 0.084 | 19.4 ± 1.5 |
| High dose | | | | | |
| (n = 8) | | | | | |
| Cilostazol Low | 5.80 ± 0.22 | 5.51 ± 0.21 | 4.26 ± 0.28 | 1.246 ± 0.115 | 22.7 ± 2.4 |
| dose (n = 8) | | | | | |
| Cilostazol Mid- | 5.70 ± 0.16 | 5.43 ± 0.14 | 4.25 ± 0.39 | 1.257 ± 0.321 | 23.0 ± 5.7 |
| dose (n = 8) | | | | | |
| Cilostazol High | 5.73 ± 0.31 | 5.47 ± 0.26 | 4.25 ± 0.42 | 1.245 ± 0.303 | 22.7 ± 5.9 |
| dose (n = 8) | | | | | |
| Methylpred- | 5.90 ± 0.92 | 5.62 ± 0.90 | 4.42 ± 0.82 | 1.054 ± 0.923 | 21.5 ± 4.7 |
| nilsolone | | | | | |
| Low dose (n = 8) | | | | | |
| Methylpred- | 5.68 ± 0.34 | 5.40 ± 0.30 | 4.16 ± 0.40 | 1.247 ± 0.136 | 23.1 ± 2.2 |
| nilsolone | | | | | |
| Mid-dose (n = 8) | | | | | |
| Methylpred- | 5.50 ± 0.26 | 5.25 ± 0.24 | 4.08 ± 0.21 | 1.165 ± 0.231 | 22.2 ± 1.4 |
| nilsolone High | | | | | |
| dose (n = 8) | | | | | |

Values represent the means ± SD.
The numbers in parentheses represent total devices.

TABLE 4
Histologic findings in 28-day Atrium Flyer stents loaded with various drugs.

| | | | | | % Struts | |
|---|---|---|---|---|---|---|
| | % struts | | | % struts | with | |
| | with | Fibrin | Endothelium | with | Giant | Inflam |
| Treatment Group | Fibrin | Score | (%) | RBC | cells | Score |
| TAXUS ™ Express | 57.3 ± 23.3 | 1.50 ± 1.00 | 75.9 ± 7.8 | 20.0 ± 7.8 | 11.1 ± 13.1 | 1.92 ± 0.69 |
| (n = 4) | | | | | | |
| Sirolimus High dose | 17.9 ± 8.6 | 0.41 ± 0.32 | 97.2 ± 5.0 | 1.2 ± 2.4 | 21.9 ± 7.6 | 1.00 ± 0.72 |
| (n = 4) | | | | | | |
| Paclitaxel Low dose | 12.2 ± 13.7 | 0.17 ± 0.31 | 88.5 ± 10.4 | 5.1 ± 7.2 | 32.5 ± 21.3 | 1.12 ± 0.77 |
| (n = 8) | | | | | | |
| Paclitaxel | 5.8 ± 8.8 | 0 | 87.9 ± 10.4 | 1.5 ± 2.5 | 28.4 ± 8.3 | 1.17 ± 0.76 |
| Mid-dose (n = 8) | | | | | | |
| Paxlitaxel | 33.9 ± 24.5 | 0.79 ± 0.85 | 85.3 ± 13.4 | 8.1 ± 15.5 | 22.6 ± 10.6 | 0.67 ± 0.36 |
| High dose (n = 8) | | | | | | |
| Cilostazol Low dose | 5.3 ± 6.2 | 0.41 ± 0.12 | 98.5 ± 2.2 | 2.6 ± 3.1 | 32.4 ± 19.4 | 1.21 ± 0.91 |
| (n = 8) | | | | | | |
| Cilostazol Mid-dose | 1.9 ± 3.8 | 0 | 97.8 ± 2.2 | 0.3 ± 0.9 | 17.8 ± 17.0 | 0.54 ± 0.59 |
| (n = 8) | | | | | | |
| Cilostazol High dose | 5.3 ± 6.2 | 0.83 ± 0.15 | 92.6 ± 8.9 | 1.4 ± 3.9 | 22.5 ± 9.5 | 0.67 ± 0.67 |
| (n = 8) | | | | | | |
| Methylprednilsolone | 3.6 ± 9.2 | 0.08 ± 0.24 | 95.1 ± 10.3 | 4.0 ± 8.4 | 16.5 ± 13.2 | 0.75 ± 0.50 |
| Low dose (n = 8) | | | | | | |
| Methylprednilsolone | 8.3 ± 10.7 | 0.08 ± 0.15 | 85.3 ± 7.3 | 3.6 ± 3.3 | 30.1 ± 9.2 | 1.04 ± 0.60 |
| Mid-dose (n = 8) | | | | | | |
| Methylprednilsolone | 1.5 ± 3.3 | 0 | 94.8 ± 7.4 | 0.7 ± 1.3 | 27.4 ± 17.9 | 0.54 ± 0.53 |
| High dose (n = 8) | | | | | | |

Values represent the means ± SD.
The numbers in parentheses represent total devices.
Abbreviations:
Endothel. = endothelium,
RBC = red blood cells,
Inflamm. = Inflammation

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the appended claims is reserved. It is intended that the present invention be limited only to the extent required by the appended claims and the applicable rules of law.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

While the present inventions have been described in conjunction with various embodiments and examples, it is not intended that the present teachings be limited to such embodiments or examples. On the contrary, the present inventions encompass various alternatives, modifications, and equivalents, as will be appreciated by those of skill in the art.

The claims should not be read as limited to the described order or elements unless stated to that effect. It should be understood that various changes in form and detail may be made without departing from the scope of the appended claims. Therefore, all embodiments that come within the scope and spirit of the following claims and equivalents thereto are claimed.

1. A method of treating vascular injury in a mammal, the method comprising locally administering:
(a) a therapeutically effective amount of a mTOR targeting compound for reducing vascular smooth muscle cell proliferation substantially at the site of administration;
(b) a therapeutically effective amount of a calcineurin inhibitor for reduction of inflammation substantially at the site of administration;
wherein the mTOR targeting compound and the calcineurin inhibitor are formulated in a bio-absorbable carrier component; and
wherein local administration comprises administration as a coating on a medical device.

2. A method of treating vascular injury in a mammal, the method comprising locally administering:
a therapeutic formulation in a therapeutically effective amount for increasing the rate of endothelial cell formation at the site of vascular injury, the therapeutic formulation comprising a mTOR targeting compound and a calcineurin inhibitor.
wherein the mTOR targeting compound and the calcineurin inhibitor are formulated in a bio-absorbable carrier component; and
wherein local administration comprises administration as a coating on a medical device.

3. A method of treating neointima proliferation and local inflammation in a mammal resulting from the injury to the interior of a vascular vessel of the mammal, comprising locally administering:
(a) a therapeutically effective amount of an mTOR targeting compound;
(b) a therapeutically effective amount of a calcineurin inhibitor;
wherein the mTOR targeting compound and the calcineurin inhibitor are formulated in a bio-absorbable carrier component; and
wherein local administration comprises administration as a coating on a medical device.

4. The method of claim 1, wherein the mTOR targeting compound comprises rapamycin, sirolimus, 40-O-(2-hydroxyethyl)-rapamycin, 40-[3-hydroxy-2-(hydroxymethyl)-2-methylpropanoate]-rapamycin (also called CC1779), 40-epi-(tetrazolyl)-rapamycin (also called ABT578), 16-pent-2-ynyloxy-32(S)-dihydrorapamycin and TAFA-93, or a compound of formula I: 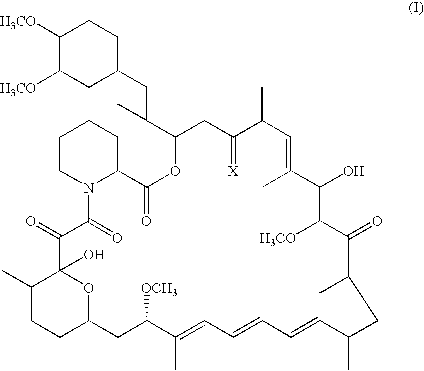
wherein
X is O, or (H,OH); or
derivatives, analogs, esters, prodrugs, pharmaceutically acceptably salts thereof, or conjugates thereof which have or whose metabolic products have the same mechanism of action.

5. The method of claim 1, wherein the calcineurin compound comprises FK506, tacrolimus, cyclosporine, a compound of formula (II), a compound of formula (III); or 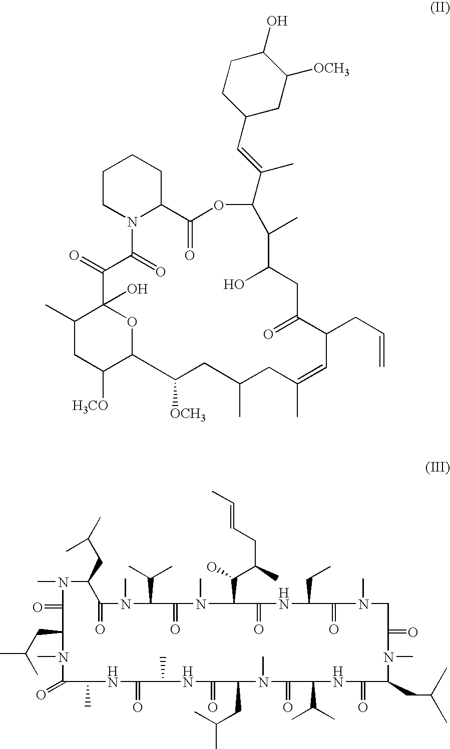
derivatives, analogs, esters, prodrugs, pharmaceutically acceptably salts thereof, or conjugates thereof which have or whose metabolic products have the same mechanism of action.

6. The method of claim 1, wherein the bio-absorbable carrier component is at least partially formed of a cellular uptake inhibitor and a cellular uptake enhancer, and the coating further including a therapeutic agent.

7. The method of claim 6, wherein the cellular uptake inhibitor comprises alpha-tocopherol.

8. The method of claim 6, wherein the cellular uptake enhancer comprises fatty acids.

9. The method of claim 6, wherein the cellular uptake enhancer comprises myristic acid added to the bio-absorbable carrier component.

10. The method of claim 1, wherein the bio-absorbable carrier component comprises at least one of a naturally occurring oil, and an oil composition.

11. The method of claim 10, wherein the bio-absorbable carrier component comprises fish oil.

12. The method of claim 1, wherein the bio-absorbable carrier component is modified from its naturally occurring state to a state of increased viscosity.

13. The method of claim 1, wherein the bio-absorbable carrier component contains omega-3 fatty acids.

14. The method of claim 1, wherein the bio-absorbable carrier is non-polymeric.

15. The method of claim 1, wherein the bio-absorbable carrier comprises a solvent.

16. The method of claim 15, wherein the bio-absorbable carrier, wherein the solvent comprises N-Methyl-2-Pyrrolidone (NMP).

17. The method of claim 1, wherein the mTOR targeting compound and the calcineurin inhibitor are substantially compartmentalized by the bio-absorbable carrier.

18. The method of claim 1, wherein the vascular injury comprises arises from one or more of biologically or mechanically mediated vascular injury.

19. The method of claim 1, wherein the vascular injury comprises protracted local inflammation arising from an mTOR targeting compound.

20-23. (canceled)

24. The method of claim 5, wherein the cyclosporin comprises naturally occurring cyclosporin, non-natural cyclosporin prepared by total- or semi-synthetic means, non-natural cyclosporin prepared by the application of modified culture techniques, cyclosporins A through Z, non-natural cyclosporin derivatives, artificial or synthetic cyclosporin derivatives, dihydrocyclosporins, derivatized cyclosporins, and cyclosporins in which variant amino acids are incorporated at specific positions within the peptide sequence.

25. The method of claim 1, wherein one or more of the mTOR targeting compound and the calcineurin inhibitor are formulated as solid particles.

26-27. (canceled)

28. The coated medical device of claim 1, wherein the medical device comprises a stent.

29. The coated medical device of claim 1, wherein the medical device comprises a catheter.

30. The coated medical device of claim 1, wherein the medical device comprises a graft.

31. The coated medical device of claim 1, wherein the medical device comprises a surgical mesh.

32. (canceled)
